(19) United States
(12) Patent Application Publication
(10) Pub. No.: US 2021/0052495 A1
(43) Pub. Date: Feb. 25, 2021

(54) Cooling Composition and Method of Use

(71) Applicant: Animal Ethics Pty Ltd, Yarra Glen (AU)

(72) Inventor: Charles Robert Olsson, Crestmead (AU)

(21) Appl. No.: 16/977,383

(22) PCT Filed: Mar. 8, 2019
(86) PCT No.: PCT/AU2019/050203

§ 371 Date: Sep. 1, 2020

(30) Foreign Application Priority Data
Mar. 9, 2018 (AU) 2018900772
Aug. 3, 2018 (AU) 2018902834

(51) Int. Cl. A61K 9/12; A61P 23/02; A61P 31/02; A61K 31/01; A61K 31/08; A61K 31/14; A61K 33/00; A61D 1/06; A61M 19/00; A61D 7/00; C09K 5/04; A61L 2/00

(52) U.S. Cl. CPC A61K 9/122; A61L 2101/32; A61P 23/02; A61P 31/02; A61K 31/01; A61K 31/08; A61K 31/14; A61K 33/00; A61D 1/06; A61M 19/00; A61D 7/00; C09K 5/04; A61L 2/0088; A61M 2250/00; A61L 2202/15; A61K 9/124

(57) ABSTRACT

A sprayable cooling composition comprising at least one refrigerant (DME) as well as at least one non-volatile antiseptic agent (cetrimide), wherein the cooling composition is capable of eliciting both a local refrigerant effect to a body surface area to which it is applied and providing antisepsis due to the non-volatile antiseptic agent remaining on the body surface area. The composition is useful for surgical and animal husbandry procedures, such as piglet castration.

This application claims priority of Australian provisional patent application number 2018900772, filed 9 Mar. 2018, and Australian provisional patent application number 2018902834, filed 3 Aug. 2018, the entire contents of which are incorporated herein by way of cross-reference.

### TECHNICAL FIELD

This invention relates to a cooling composition, its method of manufacture, and to its use in surgical procedures or animal husbandry procedures. In particular, the invention concerns a cooling composition that elicits both a local refrigerant effect and antisepsis.

### BACKGROUND ART

It will be clearly understood that, if a prior art publication is referred to herein, this reference does not constitute an admission that the publication forms part of the common general knowledge in the art in Australia or in any other country.

Vapocoolants are used in surgical and animal husbandry procedures to elicit a local refrigerant effect and to provide local anaesthesia for injections, intravenous insertions and other surgical procedures. Known vapocoolants effectively chill the skin to which they are applied, but vapocoolants do not usually and purposively contain a non-volatile antiseptic agent for killing or inhibiting the growth of microbes on the skin surface to which it is applied. For example, the vapocoolant described in U.S. Pat. No. 6,737,041 contains ethyl alcohol as a coolant which can in some circumstances function as a weak antiseptic agent, but its inclusion seems to be incidental to any antiseptic role because it is meant to immediately evaporate and hence contribute to the cooling effect. Since it is volatile, the ethyl alcohol does not remain on the skin to provide ongoing antisepsis.

Livestock, such as piglets, are routinely exposed to animal husbandry procedures such as tail docking, ear notching and castration. Although such procedures are painful, pain relief for those animals is rarely available. This is because providing pain relief is impractical, expensive or difficult.

### SUMMARY OF INVENTION

According to a 1^{st} aspect of the present invention, there is provided a cooling composition comprising:

at least one refrigerant; and

at least one non-volatile antiseptic agent.

According to a 2^{nd} aspect of the present invention, there is provided a sprayable cooling composition comprising at least one refrigerant as well as at least one non-volatile antiseptic agent, wherein the cooling composition is capable of eliciting both a local refrigerant effect to a body surface area to which it is applied and providing antisepsis due to the non-volatile antiseptic agent remaining on the body surface area.

According to a 3^{rd} aspect of the present invention, there is provided a method of cooling and providing antisepsis to a body surface of a subject, comprising the step of applying the cooling composition of the 1^{st} aspect of the invention or spraying the cooling composition of the 2^{nd} aspect of the invention onto an area of the body surface requiring cooling and antisepsis.

According to a 4^{th} aspect of the present invention, there is provided a surgical or animal husbandry procedure comprising the steps of:

1) applying the composition of the 1^{st} or 2^{nd} aspects to an area of the body's surface so as to provide a local refrigerant effect and antisepsis; and

2) carrying out a surgical step or step of an animal husbandry procedure, such as making a surgical incision into the body's surface.

According to a 5^{th} aspect of the present invention, there is provided use of at least one refrigerant and at least one non-volatile antiseptic agent in the preparation of a medicament for providing both a local refrigerant effect and antisepsis to a body surface of a subject.

According to a 6^{th} aspect of the present invention, there is provided a method of preparing a cooling composition comprising the step of combining at least one refrigerant with at least one non-volatile antiseptic agent.

According to a 7^{th} aspect of the present invention, there is provided a method of preparing a sprayable cooling composition comprising the step of combining at least one refrigerant with at least one non-volatile antiseptic agent.

According to an 8^{th} aspect of the present invention, there is provided a method for castrating a subject, said method comprising the steps of:

1) applying the composition of the 1^{st} or 2^{nd} aspects to a scrotum of the subject so as to provide a local refrigerant effect and antisepsis;

2) making a surgical incision into the scrotum;

3) applying a topical local anaesthetic and/or vasoconstrictor within the scrotum; and

4) cutting and removing testicles of the subject via the surgical incision; and optionally

5) applying a topical local anaesthetic and/or vasoconstrictor within the scrotum.

‘Refrigerant’ as used herein is a volatile liquid that evaporates on contact with the body surface and/or a pressurised gas that when contacting the body surface causes a local refrigerant effect whereby the body surface is cooled, chilled or frozen. In this way, the refrigerant can provide local anaesthesia, such as for injections, intravenous insertions, incisions and other surgical and animal husbandry procedures. Rapid evaporation of the volatile liquid from the body's surface or cold gas striking the body's surface causes a drop in temperature and results in temporary interruption of pain sensation. Preferably the body's surface, (or body's tissue or organ) is cooled to about 10° C. or below, whereby it results in an anaesthetic effect—eg. for reducing nerve sensitivity before surgery.

‘Non-volatile antiseptic agent’ as used herein is an agent that does not readily become gaseous and evaporate when applied to the body surface. Conversely, the agent will coat the body surface and/or be absorbed by the body surface.

The cooling composition can comprise any suitable type of refrigerant. The cooling composition can comprise one type of refrigerant or more than one type of refrigerant. The refrigerant can be a gas. The refrigerant can be a volatile liquid. The at least one refrigerant can be flammable or non-flammable. The cooling composition can comprise 1, 2, 3, 4, 5 or even more types of refrigerants. In some embodiments, the cooling composition can comprise a blend or mixture of two or more refrigerants. In some embodiments, the 2 or more refrigerants can either be a combination of gas and gas, volatile liquid and gas, or volatile liquid and volatile liquid.

Examples of suitable refrigerants include any one or more of the following:

a compressed gas such as an inert gas, such as nitrogen, carbon dioxide, nitrous oxide, oxygen or air;

a liquefied hydrocarbon such as methane, ethane, ethyl alcohol, propane, butane, n-butane, isobutane, pentane, isopentane, n-pentane; a mixture of 2, 3, 4 or more hydrocarbons (eg. a mixture of n-butane, isobutane and propane, or a mixture of propane and butane);

a fluorinated hydrocarbon such as trichloromonofluromethane, dichlorodifluoromethane, dichlorotetrafluroethane, 1,1,1,3,3 pentafluoropropane or 1,1,1,2 Tetrafluoroethane; liquid nitrogen;

an ether, such as dimethyl ether (DME) or methyl ethyl ether; or

a hydrofluoroalkane (HFA), such as HFA 134a (1,1,1,2,-tetrafluoroethane) or HFA 227 (1,1,1,2,3,3,3-heptafluoropropane) or a combination of these.

The cooling composition can comprise any suitable amount of refrigerant. Preferably the cooling composition comprises anywhere between approximately 10 and approximately 99.9 weight/weight (or weight/volume or volume/volume) % of refrigerant, which includes all 0.1 increments between 10 and 99.5%, including 30, 30.5, 31, 31.5 etc.

In some embodiments the cooling composition comprises between approximately 20% weight/weight and 80% weight/weight refrigerant. In some embodiments the cooling composition comprises between approximately 30% weight/weight and approximately 70% weight/weight refrigerant. In some embodiments, the cooling composition comprises approximately 30%, 35%, 40%, 45%, 50%, 55%, 60%, 65%, 70%, 75% or 80% weight/weight refrigerant. More preferably, the cooling composition comprises approximately 30%, 35%, 40%, 45%, 50%, 55%, 60%, 65%, 70%, 75% or 80% weight/weight ether, and most preferably DME.

The cooling composition can comprise any suitable type of non-volatile antiseptic agent. The cooling composition can comprise one type of non-volatile antiseptic agent or more than one type of non-volatile antiseptic agent. The at least one non-volatile antiseptic agent is preferably non-flammable. Preferably the antiseptic agent is capable of remaining on the body surface or soaking into the body surface and thus provide suitable antisepsis after the at least one refrigerant has evaporated or otherwise dissipated.

The cooling composition can comprise 1, 2, 3, 4, 5 or even more types of non-volatile antiseptic agents. In some embodiments, the cooling composition can comprise a blend or mixture of two or more antiseptic agents. In some embodiments, an antiseptic agent combination can be used that comprises a normally volatile antiseptic agent provided that its volatility is countered, lowered or eliminated by the other antiseptic agent present in the combination. The antiseptic agent is for killing or inhibiting the growth of microbes on the body surface to which it is applied.

Examples of suitable antiseptic agents include any one or more of the following: cetrimide, povidone-iodine, chlorhexidine, iodine, benzalkonium chloride, benzoic acid, nitrofurazone, benzoyl peroxide, hydrogen peroxide, hexachlorophene, phenol, resorcinol, cetylpyridinium chloride, chlorhexidine gluconate and parachoroxylenol (PCMX). Examples of preferable non-volatile antiseptic agents include quaternary ammonium salts. A preferred example is cetrimide, which is a mixture of different quaternary ammonium salts including cetrimonium bromide (CTAB).

The cooling composition can comprise any suitable amount of antiseptic agent. Preferably the cooling composition comprises anywhere between approximately 0.01 weight/weight (or weight/volume or volume/volume) % and approximately 15 weight/weight (or weight/volume or volume/volume) % of antiseptic agent, which includes all 0.01 increments between 0.01 and 15%, including 0.02, 0.03 etc.

Preferably, the cooling composition comprises approximately 1%, 1.5%, 2%, 2.5%, 3%, 3.5%, 4%, 4.5% or 5% weight/weight cetrimide. In some embodiments, the cooling composition comprises approximately 3.5% weight/weight cetrimide. In some embodiments, the cooling composition comprises approximately 2.5% weight/weight cetrimide. In some embodiments, the cooling composition comprises approximately 1.5% weight/weight cetrimide.

Although the cooling composition can be administered or applied to the body surface in any suitable way, preferably it is applied in the form of a spray, stream, mist or foam. In some embodiments the cooling composition can be delivered as an aerosol spray comprising a gaseous suspension of liquid particles. In some embodiments the cooling composition can be delivered as an aerosol mist comprising liquid particles. In some embodiments the cooling composition can be delivered as a foam, sprayable or otherwise, whereby the foam comprises gas pockets trapped in liquid. In some embodiments, the antiseptic agent can be delivered to the body surface in an almost frozen super-chilled form.

In preferred embodiments, the cooling composition is in the form of an aerosol spray, sprayable stream, sprayable mist or sprayable foam. The cooling composition can comprise or can be delivered from a pressurised spray container or can, in which case it may contain at least one propellant or may be pressurised in another way. In some embodiments the at least one refrigerant can function as the at least one propellant. The cooling composition can further comprise at least one solvent for the propellant or antiseptic, but this will depend on the nature of the propellant and antiseptic agent.

In some embodiments, an aerosol container can be partially filled with the antiseptic agent. In some embodiments, the aerosol container can be partially filled with the refrigerant, if different from the propellant. The container can be sealed and charged with the propellant until suitably pressurised.

In some embodiments, pressing an actuator button opens a valve of the container such that the propellant can force the antiseptic agent up a dip tube of the container and through the valve. The cooling composition can be applied as an aerosol mist or foam depending on the final composition. A specially articulated spray nozzle can also be used, if required. Suitable nozzles are shown in FIG. 1, for example.

Any suitable type of propellant or blend of propellants can be used. The propellant or propellant blend can be flammable or non-flammable. The propellant can be a compressed gas, soluble gas or liquefied gas. The propellant can also act as solvent, diluent, viscosity modifier or freezant.

Examples of suitable propellants include any one or more of the following:

a compressed gas such as an inert gas, such as nitrogen, carbon dioxide, nitrous oxide, oxygen or air;

a liquefied hydrocarbon such as methane, ethane, ethyl alcohol, propane, butane, n-butane, isobutane, pentane, isopentane, n-pentane; a mixture of 2, 3, 4 or more hydrocarbons (eg. a mixture of n-butane, isobutane and propane, or a mixture of propane and butane);

a fluorinated hydrocarbon such as trichloromonofluromethane, dichlorodifluoromethane, dichlorotetrafluroethane, 1,1,1,3,3 pentafluoropropane or 1,1,1,2 Tetrafluoroethane; liquid nitrogen;

an ether such as dimethyl ether (DME) or methyl ethyl ether; or

a hydrofluoroalkane (HFA) such as HFA 134a (1,1,1,2,-tetrafluoroethane) or HFA 227 (1,1,1,2,3,3,3-heptafluoropropane) or a combination of these.

The cooling composition can comprise any suitable amount of propellant. Preferably the cooling composition comprises anywhere between approximately 10 and approximately 99.9 weight/weight (or weight/volume or volume/volume) % of propellant, which includes all 0.1 increments between 10 and 99.5%, including 30, 30.1, 30.2, 30.3 etc.

In some preferred embodiments, the refrigerant is carbon dioxide or other type of compressed gas, which also functions as the propellant. In some preferred embodiments refrigerant and/or propellant is used to balance in the aerosol container, particularly when a compressed gas.

The cooling composition can contain a colourant so as to indicate where the at least one antiseptic agent has been applied to the body surface. Any suitable type of colourant can be used. Preferably the colourant is non-flammable. Alternatively, the at least one antiseptic agent itself can be coloured and provide a colourant function. Alternatively, if applied as a foam, then the foam itself may be visible and hence not require a colourant. For example, the foam might be opaque, eg. white in colour.

The colorant can be a pigment and/or dye. Suitable colorants include, for example, common food dyes or the ORCODERM®, ORCOBRITE® and ORCOFUR® lines of pigments and dyes sold by the Organic Dyestuffs Corporation. Preferably, the colourant is non-toxic and will not permanently stain the skin or animal hide or surrounding hair, fur or wool.

The cooling composition can comprise any suitable amount of colourant. In some embodiments the cooling composition comprises anywhere between approximately 0.1 and approximately 10 weight/weight (or weight/volume or volume/volume) % of colourant, which includes all 0.1 increments between 0.1 and 10%, including 0.2, 0.3 etc. In some embodiments the cooling composition comprises anywhere up to approximately 10 weight/weight (or weight/volume or volume/volume) % of colourant, which includes all decreasing 0.1 increments below 10%.

In a preferred embodiment, for example, a 5% cetrimide solution can be prepared by combining 5% weight/weight cetrimide powder with 95% weight/weight water. 70% weight/weight of the cetrimide solution can be combined with 30% weight/weight DME (which functions as a refrigerant/propellant), to produce a final cetrimide amount of 3.5%. For example, a 70% DME and 1% final cetrimide can be prepared by combining 30 ml of a 5% cetrimide solution with 70 ml DME.

Preferred embodiments of the cooling composition are shown in Table 1 below:

TABLE 1

| Refrigerant/Propellant (% w/w) | Antiseptic (% w/w) |
|---|---|
| Butane and propane blend | Cetrimide |
| Approximately 30-80, but | Approximately 1, 1.5, 2, 2.5, 3, |
| preferably 50 | 3.5, 4, 4.5 or 5, but preferably 3.5 |
| Butane, propane and isobutane blend | Cetrimide |
| Approximately 30-80, but | Approximately 1, 1.5, 2, 2.5, 3, |
| preferably 50 | 3.5, 4, 4.5 or 5, but preferably 3.5 |
| DME | Cetrimide |
| Approximately 30-80, but | Approximately 1, 1.5, 2, 2.5, 3, |
| preferably 30 | 3.5, 4, 4.5 or 5, but preferably 3.5 |
| DME | Cetrimide |
| 30-80, but preferably 50 | 1, 1.5, 2, 2.5, 3, 3.5, 4, 4.5 or 5, |
| | but preferably 2.5 |
| DME | Cetrimide |
| Approximately 30-80, but | Approximately 1, 1.5, 2, 2.5, 3, |
| preferably 30 | 3.5, 4, 4.5 or 5, but preferably 1.5 |
| DME | Cetrimide |
| Approximately 30-80, but | Approximately 1, 1.5, 2, 2.5, 3, |
| preferably 70 | 3.5, 4, 4.5 or 5, but preferably 1.5 |
| DME | Cetrimide |
| Approximately 30-80, but | Approximately 1, 1.5, 2, 2.5, 3, |
| preferably 80 | 3.5, 4, 4.5 or 5, but preferably 1 |
| | or 1.5 |

The compositions of Table 1 may or may not include a colourant (quantity to suit).

The cooling composition can be applied to the body surface for any suitable period of time. The time period will typically be between about 1 and 10 seconds, although it may be shorter or longer (eg. up to 15, 20, 25 or 30 seconds). Preferable application times include, but are not limited to, 1, 1.5, 2, 2.5, 3, 3.5, 4, 4.5, 5, 5.5, 6, 6.5, 7, 7.5, 8, 8.5, 9, 9.5 and 10 seconds.

As mentioned previously, the cooling composition can either cool, chill or freeze the body surface, but preferably cools it to a temperature from between about −20° C. up to about 10° C., including −20, −19.5, −19 . . . etc . . . 0, 0.5, 1, 1.5, 2, 2.5, 3 . . . etc . . . 7, 7.5, 8, 8.5, 9, 9.5 and 10° C. In some embodiments the cooling composition can cool the skin or other body surface to a temperature of between about 1 and 2° C. In some embodiments the cooling composition can cool the skin or other body surface to a temperature below about 9° C. or 10° C. The antiseptic agent can be delivered in an almost frozen super-chilled form, in which case it can dull nerve endings and provide an extended numbing/chilling effect.

The cooling composition can be applied to any suitable body surface. For example, it can be applied to skin of a person or animal, or to the hide of an animal. The body surface can be the surface of an internal body tissue or organ, for example. The body surface can be subcutaneous. The body surface can be with the body itself. Suitable examples include scrotal skin, testicle, ear and animal horn.

The cooling composition can be applied to any suitable type of subject. The subject can be a human. The subject can be another type of mammal or animal. The subject can be a farm animal or livestock, such as a sheep, horse, cow, goat or pig. The subject can be a companion animal, such as a cat or dog. The subject can be a laboratory animal, such as a mouse, rat or rabbit. Preferably the animal is a pig, piglet, horse, lamb or calf.

The composition can be used for an animal husbandry procedure. The procedure can be, for example, mulesing, shearing, castration, tail docking, ear tagging, de-horning, branding or marking. Preferably, the composition is used for castration whereby it is administered to the scrotum prior to the step of making a surgical incision.

Regarding the 8^{th} aspect, any suitable type of subject can be castrated. For example, the subject can be a piglet. Any suitable type of topical local anaesthetic and vasoconstrictor can be used. For example, a spray-on topical anaesthetic known as Tri-solfen™ (manufactured by Bayer) can be used to provide both local anaesthesia and vasoconstriction.

Any of the features described herein can be combined in any combination with any one or more of the other features described herein within the scope of the invention.

The reference to any prior art in this specification is not, and should not be taken as an acknowledgement or any form of suggestion that the prior art forms part of the common general knowledge.

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way.

### DESCRIPTION OF THE FIGURES

FIGS. 1 and 2 show different types of aerosol cans and nozzle delivery systems, for delivering cooling compositions according to different embodiments of the present invention.

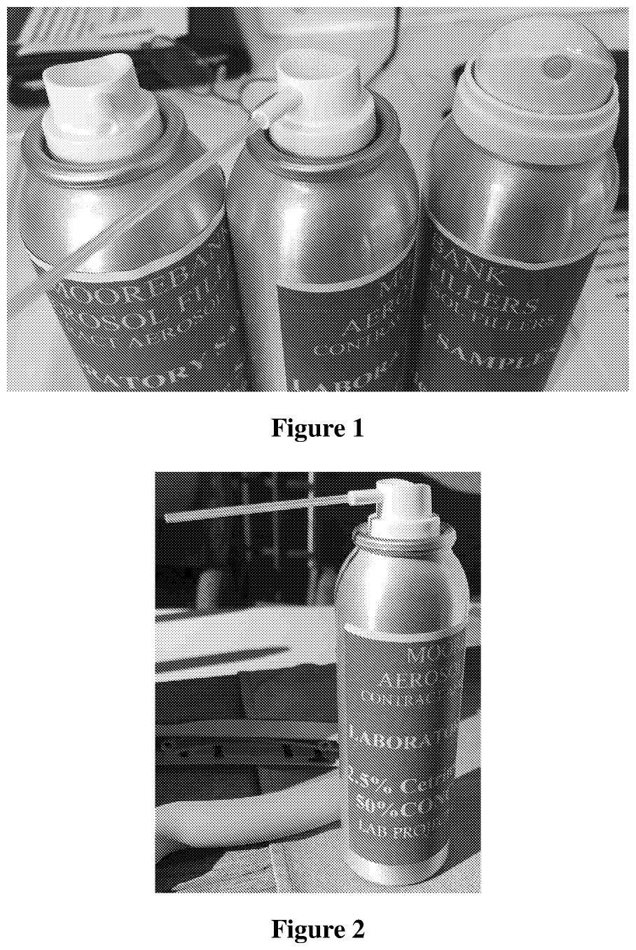

FIG. 3 shows a preferred aerosol can and nozzle delivery system for delivering a cooling composition for piglet castration. A topical anaesthetic spray called Tri-solfen™ (Bayer) that is also used for piglet castration is also shown.

FIGS. 4, 5 and 6 show different steps of a piglet castration procedure, wherein FIGS. 4 and 5 show application of the cooling composition as a white foam prior to scrotal incision, and FIG. 6 shows application of the anaesthetic/vasoconstrictor Tri-solfen™ (Bayer), according to an embodiment of the present invention.

### DETAILED DESCRIPTION

The inventor has developed a cooling composition that is capable of both cooling and providing antisepsis to a body surface of a subject.

In a first embodiment, this is achieved by way of using a compressed refrigerant/propellant gas (eg. carbon dioxide or nitrogen) and a non-volatile antiseptic agent that remains on the skin or other body surface area.

In a second embodiment, this is achieved by way of using liquefied hydrocarbon as a refrigerant/propellant and a non-volatile antiseptic agent that remains on the skin or other body surface area.

In a third embodiment, this is achieved by way of using ether as a refrigerant/propellant and a non-volatile antiseptic agent that remains on the skin or other body surface area.

In a preferred embodiment, the cooling composition is delivered from a pressurised aerosol container as a fine mist, a metered spray delivering just the right amount, or foam.

### Example 1—Preparation of a Cooling Composition Utilising Carbon Dioxide as the Refrigerant and Propellant

An aerosol container is partially filled with the liquid antiseptic agent cetrimide (5% w/w cetrimide powder+approximately 95% w/w water as a solvent) and optionally up to 5% w/w colourant in the form of a blue organic dye. The container is then sealed and charged with a suitable amount of carbon dioxide until suitably pressurised. The carbon dioxide serves both as a refrigerant and propellant. The container's composition comprises 1.0% to 5% w/w cetrimide, (optionally dye), and carbon dioxide to balance.

Pressing an actuator button opens a valve of the container such that pressurised carbon dioxide can force the (coloured) antiseptic agent up a dip tube of the container and through the valve. The cooling composition can be applied as an aerosol mist or foam depending on the final composition. A specially articulated spray nozzle can also be used, if required. See the figures for different spray nozzles that can be used.

### Example 2—Preparation of a Cooling Composition Utilising Liquified

Hydrocarbon as the Refrigerant and Propellant

An aerosol container is partially filled with the liquid antiseptic agent cetrimide (5% w/w cetrimide powder+approximately 95% w/w water as a solvent) and optionally up to 5% w/w colourant in the form of a blue organic dye. The container is then sealed and charged with hydrocarbon until suitably pressurised. The hydrocarbon serves both as a refrigerant and propellant. The container's composition comprises 1 to 5% w/w cetrimide, (optionally dye), and 50-75% w/w butane and propane blend or butane, propane and isobutane blend.

Pressing an actuator button opens a valve of the container such that pressurised hydrocarbon can force the (coloured) antiseptic agent up a dip tube of the container and through the valve. The cooling composition can be applied as an aerosol mist or foam depending on the final composition. A specially articulated spray nozzle can also be used, if required. See the figures for different spray nozzles that can be used.

### Example 3—Preparation of a Cooling Composition Utilising DME as the Refrigerant and Propellant

An aerosol container is partially filled with the liquid antiseptic agent cetrimide (5% weight/weight cetrimide powder+approximately 95% weight/weight water as a solvent) and optionally up to 5% weight/weight colourant in the form of a blue organic dye. The container is then sealed and charged with DME until suitably pressurised. DME serves both as a refrigerant and propellant. The container's composition comprises 1 to 5% w/w cetrimide, (optionally dye), and 20-80% w/w DME, preferably 1.5% cetrimide (30 ml of a 5% concentrate) and 70% DME (˜70 ml DME).

Pressing an actuator button opens a valve of the container such that pressurised DME can force the (coloured) antiseptic agent up a dip tube of the container and through the valve. The cooling composition can be applied as an aerosol mist or foam depending on the final composition. A specially articulated spray nozzle can also be used, if required. See the figures for different spray nozzles that can be used.

### Example 4—Preferred Cooling Composition Formulations

A 5% cetrimide solution was prepared by combining 5% w/w cetrimide powder with approximately 95% w/w water. A select quantity of the cetrimide solution was then combined with the refrigerant/propellant.

Preferred aerosol formulations are shown in Table 1 below.

TABLE 1
Preferred Aerosol Formulations

| Formulation | Refrigerant/Propellant | Antiseptic | Dye (optional) |
|---|---|---|---|
| No. | (% w/w) | (% w/w) | (% w/w) |
| 1 | Butane and propane | Cetrimide | Blue organic dye |
| | blend | 3.5 | Quantity to suit |
| | 50 | | |
| 2 | Butane, propane and | Cetrimide | Blue organic dye |
| | isobutane blend | 3.5 | Quantity to suit |
| | 50 | | |
| 3 | DME | Cetrimide | Blue organic dye |
| | 30 | 3.5 | Quantity to suit |
| 4 | DME | Cetrimide | Blue organic dye |
| | 50 | 2.5 | Quantity to suit |
| 5 | DME | Cetrimide | Blue organic dye |
| | 30 | 1.5 | Quantity to suit |
| 6 | DME | Cetrimide | Blue organic dye |
| | 70 | 1.5 | Quantity to suit |
| 7 | DME | Cetrimide | Blue organic dye |
| | 80 | 1 (or 1.5) | Quantity to suit |

Pressing an actuator button opens a valve of the container such that pressurised propellant can force the (coloured) antiseptic agent up a dip tube of the container and through the valve. The cooling composition can be applied as an aerosol mist or foam depending on the final composition. A specially articulated spray nozzle can also be used, if required. See the figures for different spray nozzles that can be used.

### Example 5—Use of the Cooling Compositions

The cooling composition of the Examples is sprayed onto a (human or animal) subject's skin/hide for the required period of time so as to elicit cooling and hence a local anaesthetic effect. Although the refrigerant dissipates, the antiseptic agent remains on the skin/hide to provide prolonged antisepsis. Successful application of the cooling composition can be confirmed by way of it including the colourant—if the antiseptic agent itself is not opaque/visible enough.

After applying the cooling composition, the animal or human can be subjected to surgery or an animal husbandry procedure, such as castration, dehorning or ear tagging. For example, the cooling composition can be used to cool the scrotum of an animal prior to incision.

The cooling composition of the Examples can be sprayed onto other different body surfaces in a similar manner, including surfaces of tissues or internal organs.

### Example 6—Use of a Cooling Composition on Skin

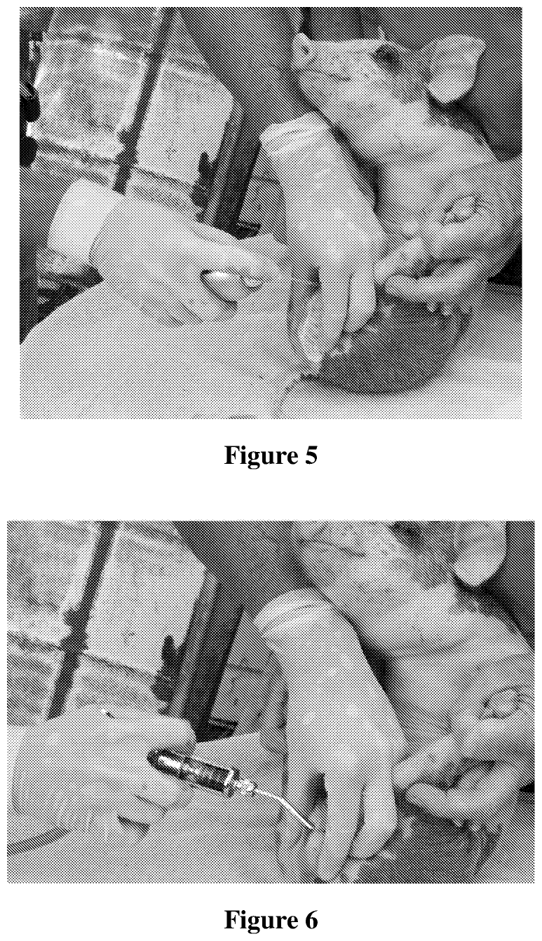

Cooling composition, eg. 50% (w/w) DME and 2.5% (w/w) cetrimide supplied in an approximately 100 mL pressure-pack, was applied directly to skin of a subject. See FIG. 4. Spray application was from approximately 8 cm (nozzle to skin surface). Application was over approximately forty (40) seconds in three equal bursts, each of 3 seconds with a ten (10) second delay between applications.

A (subcutaneous) skin temperature equal to or below 10 degrees Celsius was achieved following the third spray application. This temperature was retained for approximately 10 seconds, thereby allowing adequate time for skin incision or other procedure.

### Example 7—Use of a Cooling Composition for Piglet Castration

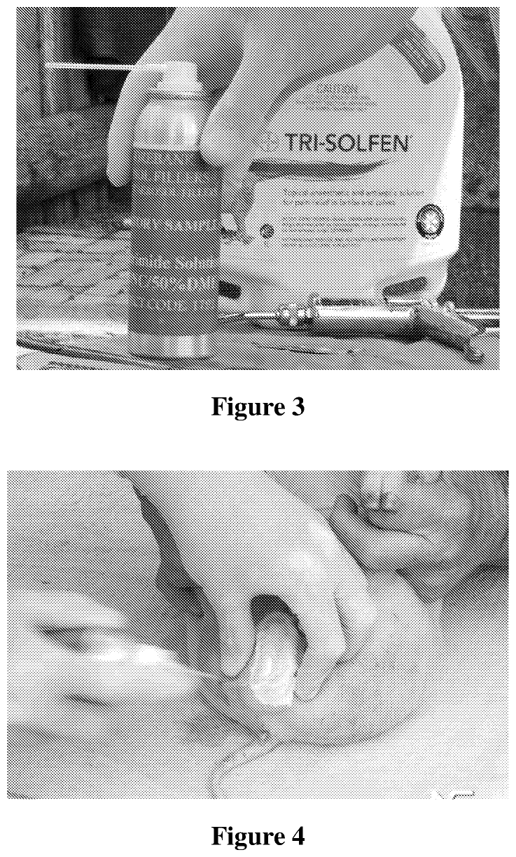

Cooling composition, eg. 50% (w/w) DME and 2.5% (w/w) cetrimide, supplied in an approximately 100 mL pressure-pack (see FIG. 3), was applied directly to scrotal skin of 3 to 7-day old piglets (see FIG. 4). Spray application was from approximately 8 cm (nozzle to skin surface). Application was over approximately forty (40) seconds in three equal bursts, each of 3 seconds with a ten (10) second delay between applications.

Subcutaneous (directly under the scrotal skin) temperature was monitored using a temperature probe. A skin temperature equal to or below 10 degrees Celsius was achieved following the third spray application. This temperature was retained for approximately 10 seconds, thereby allowing adequate time for skin incision. Subsequent incision of the skin within this 10 second timeframe appeared to result in less discomfort to the animal as reflected by vocalisation and body responses.

### Example 8—Use of a Cooling Composition for Piglet Castration

Piglets of appropriate age were selected and, once restrained, cooling composition (50% (w/w) DME and 2.5% (w/w) cetrimide) as shown in FIG. 3 was sprayed directly over the body area where an incision for castration was to be made. See FIGS. 5 and 6. Because the chilling factor reduced any pain transmitted through nerve endings, the impact of the incision was less as far as the pain response was concerned. After waiting two or three seconds, a single incision was made over the middle of the scrotum. A spray-on topical anaesthetic known as Tri-solfen™ was then sprayed into the area and its local anaesthetic was given 20 seconds to work. See FIG. 6. The Tri-solfen™ anaesthetic was used in this area because it had contact with the blood vessels and nerves that are associated with the testicle. Following that, two further incisions were made to remove the testicles through the incision site. After cutting and removing the testicles, the Tri-solfen™ anaesthetic spray was applied to both provide further anaesthesia and constrict blood vessels by way of Tri-solfen™'s vasoconstrictor/adrenaline.

The treated piglets did not kick or squeal during the procedure, and once the procedure was over they typically returned to suckle from their mother.

Cooling down the actual tactile nature of cetrimide led to proper skin and nerve desensitization, compared with just using a basic vapocoolant.

### Example 9—Particularly Preferred Aerosol Formulations

A particularly preferred formulation has a very high percentage of DME, so as to deliver super-chilled cetrimide. A preferred formulation is 70% (w/w) DME and 1.5% (w/w) cetrimide. The formulation could contain as much as 80% DME, provided that the cetrimide (eg. 1 to 2%) can still be dispensed from a pressurised can.

Advantages of the present invention as exemplified include:

The cooling composition can be easily applied to a subject, such as an animal.

The cooling composition can be easily applied to a large number of animals in a short period of time.

Pain relief can be provided without injection or other invasive technique.

Successful application of the cooling composition can be identified by way of the colourant (if present).

The cooling composition can be applied in a metered dose and different dosages can result in different degrees of body surface cooling.

A single application can provide both pain relief and ongoing antisepsis.

The inclusion of cetrimide led to proper skin and nerve desensitization.

A high percentage of DME can deliver super-chilled cetrimide, so as to improve or prolong the numbing effect.

In the present specification (and claims), the word ‘comprising’ and its derivatives including ‘comprises’ and ‘comprise’ include each of the stated integers but does not exclude the inclusion of one or more further integers.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims (if any) appropriately interpreted by those skilled in the art.

1.-20. (canceled)

21. A sprayable cooling composition comprising at least one refrigerant as well as at least one non-volatile antiseptic agent, wherein the cooling composition is capable of eliciting both a local refrigerant effect to a body surface area to which it is applied and providing antisepsis due to the non-volatile antiseptic agent remaining on the body surface area.

22. The composition of claim 21, wherein the cooling composition comprises anywhere between approximately 20% (w/w) and approximately 80% (w/w) of said at least one refrigerant.

23. The composition of claim 21, wherein the cooling composition comprises anywhere between approximately 1% and approximately 5% weight/weight of said at least one non-volatile antiseptic agent.

24. The composition of claim 21, wherein the at least one refrigerant comprises one or more refrigerants selected from the group consisting of:
a compressed gas;
a liquefied hydrocarbon;
a fluorinated hydrocarbon;
an ether; and
a hydrofluoroalkane.

25. The composition of claim 21, wherein the at least one refrigerant is dimethyl ether (DME).

26. The composition of claim 21, wherein the at least one non-volatile antiseptic agent is one or more antiseptic agents selected from the group consisting of: cetrimide; povidone-iodine; chlorhexidine; iodine; benzalkonium chloride; benzoic acid; nitrofurazone; benzoyl peroxide; hydrogen peroxide; hexachlorophene; phenol; resorcinol; cetylpyridinium chloride; chlorhexidine gluconate; and parachoroxylenol.

27. The composition of claim 21, wherein the at least one non-volatile antiseptic agent comprises at least one quaternary ammonium salt.

28. The composition of claim 27, wherein the at least one non-volatile antiseptic agent comprises cetrimide.

29. The composition of claim 21, wherein the cooling composition further comprises a colorant.

30. The composition of claim 21, wherein the cooling composition is formulated to be applied to the body surface in a form selected from the group consisting of: a spray; stream; mist; and foam.

31. The composition of claim 30, wherein the cooling composition is in the form of an aerosol spray.

32. The cooling composition of claim 21, comprising a formulation selected from the group consisting of:
approximately 20-80% (w/w) butane and propane blend, and approximately 1.0-5.0% (w/w) cetrimide;
approximately 20-80% (w/w) butane, propane and isobutane blend, and approximately 1.0-5.0% (w/w) cetrimide;
approximately 50% (w/w) butane and propane blend, and approximately 3.5% (w/w) cetrimide;
approximately 50% (w/w) butane, propane and isobutane blend, and approximately 3.5% (w/w) cetrimide;
approximately 20%-80% (w/w) DME, and approximately 1.0-5.0% (w/w) cetrimide;
approximately 30% (w/w) DME, and approximately 3.5% (w/w) cetrimide;
approximately 50% (w/w) DME, and approximately 2.5% (w/w) cetrimide;
approximately 30% (w/w) DME, and approximately 1.5% (w/w) cetrimide;
approximately 70% (w/w) DME, and approximately 1.5% (w/w) cetrimide; and
approximately 80% (w/w) DME, and approximately 1.0% (w/w) cetrimide.

33. A method of cooling and providing antisepsis to a body surface of a subject, comprising the step of applying the cooling composition of claim 21 to an area of the body surface requiring cooling and antisepsis.

34. The method of claim 33, wherein the cooling composition comprises a formulation selected from the group consisting of:
approximately 20-80% (w/w) butane and propane blend, and approximately 1.0-5.0% (w/w) cetrimide;
approximately 20-80% (w/w) butane, propane and isobutane blend, and approximately 1.0-5.0% (w/w) cetrimide;
approximately 50% (w/w) butane and propane blend, and approximately 3.5% (w/w) cetrimide;
approximately 50% (w/w) butane, propane and isobutane blend, and approximately 3.5% (w/w) cetrimide;
approximately 20%-80% (w/w) DME, and approximately 1.0-5.0% (w/w) cetrimide;
approximately 30% (w/w) DME, and approximately 3.5% (w/w) cetrimide;
approximately 50% (w/w) DME, and approximately 2.5% (w/w) cetrimide;
approximately 30% (w/w) DME, and approximately 1.5% (w/w) cetrimide;
approximately 70% (w/w) DME, and approximately 1.5% (w/w) cetrimide; and
approximately 80% (w/w) DME, and approximately 1.0% (w/w) cetrimide.

35. A surgical or animal husbandry procedure comprising the steps of:
1) applying the cooling composition of claim 21 to an area of a body's surface so as to provide a local refrigerant effect and antisepsis; and
2) carrying out a surgical step or step of an animal husbandry procedure.

36. The procedure of claim 35, wherein the cooling composition comprises a formulation selected from the group consisting of:
approximately 20-80% (w/w) butane and propane blend, and approximately 1.0-5.0% (w/w) cetrimide;
approximately 20-80% (w/w) butane, propane and isobutane blend, and approximately 1.0-5.0% (w/w) cetrimide;
approximately 50% (w/w) butane and propane blend, and approximately 3.5% (w/w) cetrimide;
approximately 50% (w/w) butane, propane and isobutane blend, and approximately 3.5% (w/w) cetrimide;
approximately 20%-80% (w/w) DME, and approximately 1.0-5.0% (w/w) cetrimide;
approximately 30% (w/w) DME, and approximately 3.5% (w/w) cetrimide;
approximately 50% (w/w) DME, and approximately 2.5% (w/w) cetrimide;
approximately 30% (w/w) DME, and approximately 1.5% (w/w) cetrimide;
approximately 70% (w/w) DME, and approximately 1.5% (w/w) cetrimide; and
approximately 80% (w/w) DME, and approximately 1.0% (w/w) cetrimide.

37. The procedure of claim 35, wherein the surgical or animal husbandry procedure is castration and the composition is applied to a scrotum of the subject so as to provide a local refrigerant effect and antisepsis.

38. The procedure of claim 35, comprising the steps of:
1) applying a sprayable cooling composition, comprising at least one refrigerant as well as at least one non-volatile antiseptic agent, wherein the cooling composition is capable of eliciting both a local refrigerant effect to a body surface area to which it is applied and providing antisepsis due to the non-volatile antiseptic agent remaining on the body surface area, to a scrotum of the subject so as to provide a local refrigerant effect and antisepsis;
2) making a surgical incision into the scrotum;
3) applying a topical local anaesthetic and/or vasoconstrictor within the scrotum; and
4) cutting and removing testicles of the subject via the surgical incision; and optionally
5) applying a topical local anaesthetic and/or vasoconstrictor within the scrotum.

39. The procedure of claim 37, wherein said subject is a piglet.
